(19) United States
(12) Patent Application Publication
(10) Pub. No.: US 2005/0159649 A1
(43) Pub. Date: Jul. 21, 2005

(54) Laryngoscope and associated method of intubating a patient

(76) Inventor: Rajeev J. Patel, Plano, TX (US)

Correspondence Address: KONNEKER & SMITH P. C., 660 NORTH CENTRAL EXPRESSWAY, SUITE 230, PLANO, TX, 75074, US

(21) Appl. No.: 11/031,636

(22) Filed: Jan. 7, 2005

Related U.S. Application Data
(60) Provisional application No. 60/537,150, filed on Jan. 16, 2004.

(51) Int. Cl. A61B 1/267

(52) U.S. Cl. 600/194; 600/190

(57) ABSTRACT

A laryngoscope and associated method of intubating a patient. A laryngoscope includes a handle and a blade extending outwardly from the handle. The blade has a surface for contacting a tongue of a patient during an intubation procedure. The blade surface includes first, second and third sections. The first section is positioned between the handle and the second section. The second section is positioned between the first and third sections. The first and third sections are concave. The second section is convex. A method of intubating a patient includes the steps of: inserting a laryngoscope into an oral cavity, the laryngoscope including a blade having a surface for contacting a tongue; displacing the tongue into a submental space using a first concave section of the blade surface; and preventing subluxation of the tongue into a pharyngeal cavity using a second convex section of the blade surface.

### CROSS-REFERENCE TO RELATED APPLICATION

The present application claims the benefit of the filing date of provisional U.S. patent application no. 60/537,150 filed Jan. 16, 2004. The entire disclosure of the provisional application is incorporated herein by this reference.

### BACKGROUND

The present invention relates generally to equipment and methods utilized in medical procedures and, in an embodiment described herein, more particularly provides a laryngoscope and associated method of intubating a patient.

In the process of inserting a tube into the trachea of a patient, a device known as a laryngoscope is typically used to aid in opening up a passage for the tube and observing the passage. Unfortunately, most existing laryngoscopes and methods for using the laryngoscopes are deficient in several aspects.

For example, some laryngoscopes visually obstruct the passage, thereby making it difficult to observe the passage, so that the medical personnel intubating the patient cannot tell whether the passage is clear, aligned, or otherwise ready for inserting an intubation tube in the trachea. Some laryngoscopes are provided with a light source to aid in illuminating the passage, but typically these light sources include battery-powered incandescent bulbs and are not of disposable design, so that the batteries and bulb must be periodically replaced and the laryngoscope must be sterilized after each use.

Some laryngoscopes do not adequately align the passage for the tube, thereby making it difficult to insert the tube into the trachea. One typical deficiency in this respect is a failure to maintain control of the tongue. One result of this can be subluxation of the tongue into the pharyngeal cavity, obstructing the view or the passage for the tube into the tracheal ostea. Another deficiency is the lack of manipulation of the tongue in the oral cavity by other laryngoscopes which can obstruct the view of the tracheal ostea.

Therefore, it may be seen that improvements are needed in the art of laryngoscopes and associated methods. It is an object of the present invention to provide such improvements.

### SUMMARY

In carrying out the principles of the present invention, a laryngoscope and associated method of intubating a patient are provided which solve at least one problem in the art. An example is described below in which the laryngoscope is a disposable unit having an integrally-formed handle and blade, and a self-contained light source.

In one aspect of the invention, a laryngoscope is provided which includes a handle and a blade extending outwardly from the handle. The blade has a surface for contacting a tongue of a patient during an intubation procedure. The blade surface includes first, second and third sections. The first section is positioned between the handle and the second section. The second section is positioned between the first and third sections. The first and third sections are concave. The second section is convex.

In another aspect of the invention, a method of intubating a patient includes the steps of: inserting a laryngoscope into an oral cavity of the patient, the laryngoscope including a blade having a surface for contacting a tongue of the patient; displacing the tongue into a submental space of the patient using a first concave section of the blade surface; and preventing subluxation of the tongue into a pharyngeal cavity of the patient using a second convex section of the blade surface.

These and other features, advantages, benefits and objects of the present invention will become apparent to one of ordinary skill in the art upon careful consideration of the detailed description of a representative embodiment of the invention hereinbelow and the accompanying drawings.

### BRIEF DESCRIPTION OF THE DRAWINGS

FIG. 1 is a cross-sectional view of a method of intubating a patient, the method embodying principles of the present invention;

FIG. 2 is an enlarged scale side elevational view of a laryngoscope which may be used in the method of FIG. 1, the laryngoscope embodying principles of the present invention;

FIG. 3 is an end elevational view of the laryngoscope of FIG. 2; and

FIG. 4 is an enlarged scale view of a light source of the laryngoscope of FIG. 2.

### DETAILED DESCRIPTION

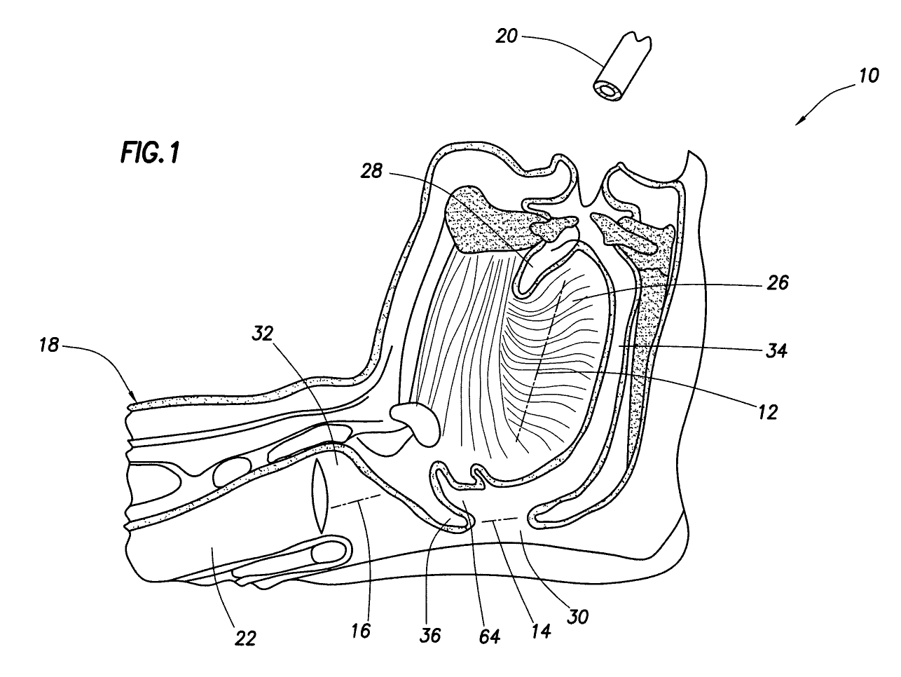

Representatively illustrated in FIG. 1 is a patient intubating method 10 which embodies principles of the present invention. In the following description of the method 10 and other apparatus and methods described herein, directional terms, such as “above”, “below”, “upper”, “lower”, etc., are used for convenience in referring to the accompanying drawings. Additionally, it is to be understood that the various embodiments of the present invention described herein may be utilized in various orientations, such as inclined, inverted, horizontal, vertical, etc., and in various configurations, without departing from the principles of the present invention. The embodiments are described merely as examples of useful applications of the principles of the invention, which is not limited to any specific details of these embodiments.

In the method 10, three anatomical axes 12, 14, 16 of a patient 18 are aligned to facilitate insertion of a tube 20 into a trachea 22 of the patient. The axis 12 is the oral axis extending from the mouth opening to the pharynx along the oral cavity 34. The axis 14 is the pharyngeal axis extending along the pharyngeal cavity 30. The axis 16 is the laryngeal axis extending along the laryngeal cavity 32.

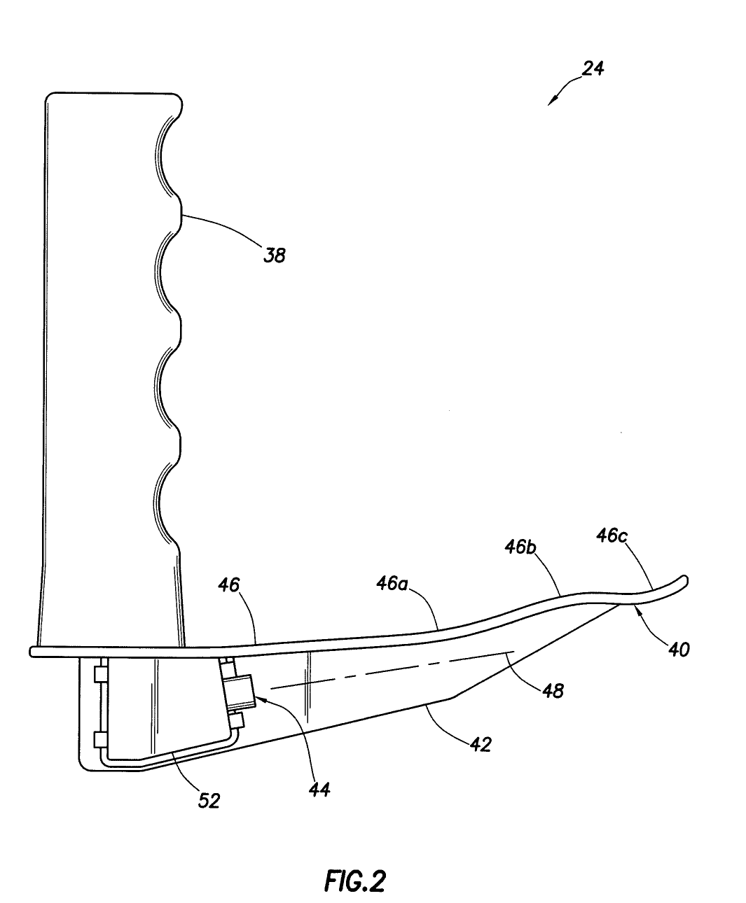

A laryngoscope 24 (shown in FIGS. 2-4) of the invention functions to better align the axes 12, 14, 16. The laryngoscope 24 beneficially displaces the tongue 26 into the submental space 28 and maintains control of the tongue, preventing subluxation of the tongue into the pharyngeal cavity 30. The soft tissue of the pharynx is also displaced by the laryngoscope 24 to permit enhanced observation of the tracheal lumen. A tip of a blade of the laryngoscope 24 may rest in the vallecular area 64, or it may be used to lift the epiglottis 36 if needed to permit enhanced observation of the tracheal lumen.

A light source of the laryngoscope 24 is conveniently positioned to illuminate the passage for the tube 20. The laryngoscope 24 is preferably constructed of clear or translucent materials (such as polycarbonate, etc.) to further enhance the illumination of the passage. In addition, a blade of the laryngoscope 24 may be clear to permit viewing the epiglottis 36 through the blade.

Referring additionally now to FIG. 2, an enlarged scale side view of the laryngoscope 24 is representatively illustrated. In this view it may be seen that the laryngoscope 24 includes a handle 38, a blade 40, a supporting rib 42 and a light source 44. In use, the handle 38 is grasped by a user and an upper surface 46 of the blade 40 is pressed against the tongue 26 and possibly epiglottis 36 of the patient 18 as described in further detail below, to thereby facilitate aligning the three axes 12, 14, 16 in the method 10.

The surface 46 of the blade 40 preferably includes three curved sections 46a, 46b, 46c. The section 46a is concave facing the tongue 26 of the patient 18. This shape allows the tongue 26 to be displaced into the submental space 28 while maintaining control of the tongue (i.e., the tongue will be naturally biased toward the concave depression formed by the surface 46a).

The section 46b is convex facing the tongue 26 of the patient 18. When appropriately positioned in the oral cavity 34, the section 46b will be located at the rear of the tongue 26 and, when pressed against the tongue, will function to prevent subluxation of the tongue into the pharyngeal cavity 30. This portion of the blade 40 also outwardly displaces the soft tissue bounding the pharyngeal cavity 30, permitting better observation of the tracheal lumen.

The section 46c is concave facing the tongue 26 of the patient 18. This section 46c may be used to lift the epiglottis 36 to better observe the tracheal lumen. Alternatively, this portion of the blade 40 may rest in the vallecular area 64 at the rear of the tongue 26.

The rib 42 supports the blade 40 as it is pressed against the tongue 26. Note that the rib 42 extends from the handle 38 portion to a position between the two sections 46b, 46c on the blade 40. This provides substantial support to the portion of the blade 40 having the section 46a thereon which is pressed directly against the tongue 26.

The light source 44 is attached to an inner side of the rib 42. The light source 44 is not obstructed by the blade 40, and can direct light along an axis 48 which is aligned with the anatomical axes 12, 14, 16. If the blade 40 is made of a transparent material, this illumination may also be used to observe the tongue 26 and epiglottis 36 through the blade.

Preferably, the handle 38, blade 40 and rib 42 are integrally constructed of a single piece of material and are disposable, being used only once. One advantage to the laryngoscope 24 being disposable is that the time and expense of sterilizing the laryngoscope after each use is eliminated. However, it should be clearly understood that it is not necessary for the laryngoscope 24 to be disposable. The laryngoscope 24 could instead be reusable, in which case the integral construction of the handle 38, blade 40 and rib 42 will eliminate the possibility that the blade might be sterilized without the handle also being sterilized (e.g., as could happen with prior non-integrally constructed laryngoscopes).

If the laryngoscope 24 is disposable, the light source 44 is also preferably a self-contained unit in which no replacement of batteries or bulb is provided for. However, many alternatives can be used for the light source 44. If the laryngoscope 24 is reusable, then the batteries and bulb could be conveniently replaceable. As another alternative, the light source 44 could have the batteries positioned in the handle 38, with the bulb either in the handle or attached to the rib 42.

If the bulb is in the handle 38, a fiber optic line or other light conduit may be used to direct the light from the handle to a desired position on or adjacent the rib 42. Light may also be transmitted directly by the material of the laryngoscope 24 itself. For example, the blade 40 and/or rib 42 may be made of a transparent or translucent material to transmit light from the light source 44 to illuminate the oral cavity 34, pharyngeal cavity 30 and laryngeal cavity 32.

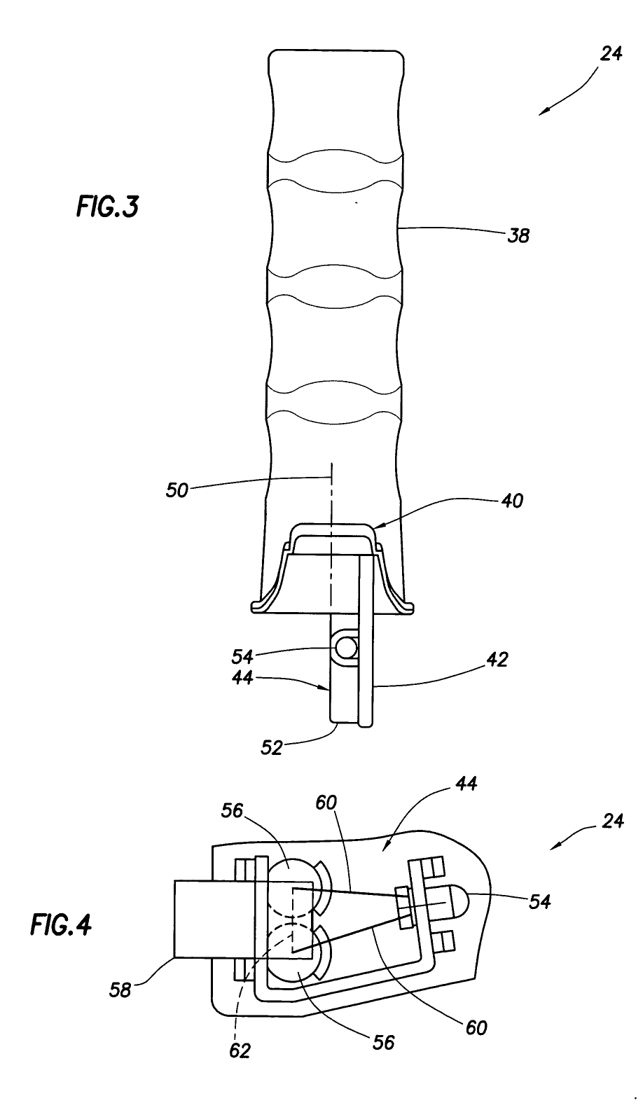

Referring additionally now to FIG. 3, an end view of the laryngoscope 24 is depicted. In this view it may be seen that the rib 42 is laterally offset from a center axis 50 of the blade 40. This permits enhanced access for inserting the tube 20 while the laryngoscope 24 is in the oral cavity 34.

Note that the light source 44 is positioned toward the blade axis 50 from the rib 42. This permits the light source 44 to effectively illuminate the oral, pharyngeal and laryngeal cavities 34, 30, 32 without obstruction from the rib 42. However, if the rib 42 is made of a transparent or translucent material, light may be transmitted from the light source 44 through the rib.

Referring additionally now to FIG. 4, an enlarged side view of the light source 44 is shown with a cover 52 removed. In this view it may be seen that the light source 44 includes a bulb 54 and batteries 56.

The bulb 54 is preferably a high intensity light emitting diode (LED) which consumes relatively little electrical power. Other types of illuminating bulbs (such as incandescent, fluorescent, etc.) may be used in place of the bulb 54, if desired.

A switch in the form of a small strip of insulating material 58 is used to prevent the bulb 54 from illuminating until just prior to using the laryngoscope 24. The material 58 is withdrawn from the light source 44, thereby allowing wire leads 60 of the bulb 54 to contact the batteries 56 and thus supply electrical power to the bulb. A wire contact 62 is used to electrically connect opposite sides of the batteries 56.

Note that, in this disposable embodiment of the laryngoscope 24, the light source 44 cannot be turned off. After withdrawing the material 58, the bulb 54 will continue to illuminate until the batteries 56 are discharged. However, if the laryngoscope 24 is to be reused, a switch which may be turned off can be used in place of the material 58.

Of course, a person skilled in the art would, upon a careful consideration of the above description of representative embodiments of the invention, readily appreciate that many modifications, additions, substitutions, deletions, and other changes may be made to these specific embodiments, and such changes are within the scope of the principles of the present invention. Accordingly, the foregoing detailed description is to be clearly understood as being given by way of illustration and example only, the spirit and scope of the present invention being limited solely by the appended claims and their equivalents.

1. A laryngoscope, comprising:
a handle; and
a blade extending outwardly from the handle, the blade having a surface for contacting a tongue of a patient during an intubation procedure, the surface including first, second and third sections, the first section being positioned between the handle and the second section, the second section being positioned between the first and third sections, the first and third sections being concave, and the second section being convex.

2. The laryngoscope of claim 1, further comprising a supporting rib attached to the blade on a side of the blade opposite the blade surface.

3. The laryngoscope of claim 2, wherein the rib extends from a location proximate the handle to a location between the second and third blade surface sections.

4. The laryngoscope of claim 1, wherein the handle and blade are integrally formed of a single piece of material.

5. The laryngoscope of claim 1, further comprising a light source.

6. The laryngoscope of claim 5, wherein the light source is attached to the blade opposite the handle.

7. The laryngoscope of claim 5, wherein the light source is a self-contained unit attached to a supporting rib, the rib being attached to the blade opposite the handle.

8. The laryngoscope of claim 7, wherein the light source is positioned closer than the rib to a center axis of the blade.

9. The laryngoscope of claim 1, wherein the blade is made of a transparent material.

10. The laryngoscope of claim 1, wherein the blade is made of a translucent material.

11. A method of intubating a patient, the method comprising the steps of:
inserting a laryngoscope into an oral cavity of the patient, the laryngoscope including a blade having a surface for contacting a tongue of the patient;
displacing the tongue into a submental space of the patient using a first concave section of the blade surface; and
preventing subluxation of the tongue into a pharyngeal cavity of the patient using a second convex section of the blade surface.

12. The method of claim 11, further comprising the step of positioning a third concave section of the blade surface in a vallecular area of the patient, the second blade surface section being positioned between the first and third blade surface sections.

13. The method of claim 11, further comprising the step of displacing an epiglottis of the patient using a third concave section of the blade surface, the second blade surface section being positioned between the first and third blade surface sections.

14. The method of claim 11, further comprising the step of displacing tissue bounding a pharyngeal cavity of the patient using the blade at the second blade surface section.

15. The method of claim 11, further comprising the step of illuminating an oral cavity of the patient using a light source of the laryngoscope.

16. The method of claim 15, wherein in the illuminating step the light source is a self-contained unit attached to the blade.

17. The method of claim 15, wherein in the illuminating step, the light source includes a bulb and at least one battery, the bulb and battery being carried on a supporting rib for the blade.

18. The method of claim 11, further comprising the step of observing the epiglottis through the blade.

19. The method of claim 11, wherein the blade is at least one of transparent and translucent.

20. The method of claim 11, wherein in the inserting step the laryngoscope further includes a handle, the blade and handle being integrally formed.
